(19) United States
(12) Plant Patent Application Publication
(10) Pub. No.: US 2004/0168241 P1
(43) Pub. Date: Aug. 26, 2004

(54) Artichoke plant named BCP 20-18

(76) Inventor: Medhat Chahbandar, Torreilles (FR)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/371,882

(22) Filed: Feb. 20, 2003

(51) Int. Cl. A01H 5/00

(52) U.S. Cl. PLT/258

(57) ABSTRACT

A new and distinct cultivar of Artichoke plant named ‘BCP 20-18”. This new variety is characterized by a compact shape, a thicker heart, and it matures significantly earlier than other varieties. It has more green (less yellow) overall coloration.

### BACKGROUND OF THE INVENTION

The present invention comprises a new and distinct cultivar of artichoke plant, botanically known as Cynara scolymus L., herein referred to by the cultivar name ‘BCP 20-18’. This new variety is characterized by a compact shape with a little pyramidal in the center, a thick heart, a darker and more uniform green color (specifically, showing less yellow coloration than the parents), and it matures significantly earlier than the parent.

Cynara scolymus L., commonly known as Globe artichoke, is a perennial herb and is a member of the family Asteraceae, also known as the Compositae family. Globe artichokes comprise: leaves, which are pinnately lobed, but primarily spineless; globose capitula composed of overlapping layers of large involucral bracts; and receptacles, which are enlarged and fleshy. Globe artichoke plants are essentially grown for the production of the immature flower heads (or buds), which are considered vegetable delicacies. Fresh artichokes may be steamed, boiled, or baked after which the fleshy receptacle, inner and outer bracts, and parts of the floral stem may be eaten.

The new cultivar is a product of a planned breeding program carried out by the inventor, M. Chanbandar near Perpignan, France. It was selected from a hybrid cross between the artichoke variety BCL 9-11 and the artichoke variety BH 35-28. The inventor performed asexual reproduction of the new cultivar via division. It was demonstrated that the combination of characteristics as herein disclosed for the new cultivar are firmly fixed and retained through successive generations of asexual reproduction.

### BRIEF SUMMARY OF THE INVENTION

The following traits have been repeatedly observed and are determined to be basic characteristics of ‘BCP 20-18’. These traits in combination distinguish the Artichoke as a new and distinct cultivar from its parent(s) and other varieties known to the breeder. ‘BCP 20-18’ has a more compact shape, with a little pyramidal in the center, a thick heart, a darker and more uniform green color (specifically, showing less yellow coloration than the parents), and it matures significantly earlier than the parent.

‘BCP 20-18’ has not been observed under all possible environmental conditions. The phenotype of the new cultivar may vary with variations in environment such as temperature, humidity, light intensity and day length, without any change in the genotype. However, the following observations, measurements and values, except for seed observations, describe the new cultivar as grown in Lompoc, Calif. under conditions which closely approximate those generally used in horticultural practice. Seed observations were carried out in Perpignan, France.

Lompoc is located on California's central coast in Santa Barbara County. Conditions can vary in the summer months. Air temperatures can range between the low 60's to 80's. The relative humidity is generally high. Prevailing winds are northwesterly and rainfall averages 12 inches per year.

In the following description, holding quality was measured by the physical appearance of the head. This includes the head's appearance following 3 and 7 day storage periods in cold storage at 34° F. The head's exterior (oxidation) was observed at each of the two observation points. Browning and blackening of plant tissue was evaluated as light, moderate and extreme. Overall storage response was measured by observing heads following 3 and 7 day cold storage periods. These observations concentrated on visible color variability and/or presence of lesions or other cosmetic anomalies. Leaf ratio (L/W) was determined by dividing representative leaf sample lengths by their width. Finally, head response (weather) was determined by observing the heads at maturity. These field observations focus on the presence or absence of bronzing, necrotic or chlorotic lesions or any abiotic responses to environmental conditions. AFLP (DNA analysis) was conducted on each parental selection).

### BRIEF DESCRIPTION OF THE DRAWINGS

This new variety is illustrated by the accompanying photographic drawings, with such colors in the photograph being as true to those of the plant as can be reasonably obtained from conventional photographic procedures. In such photographs:

FIG. 1 shows a side elevational view of ‘BCP 20-18’s fruit in full color.

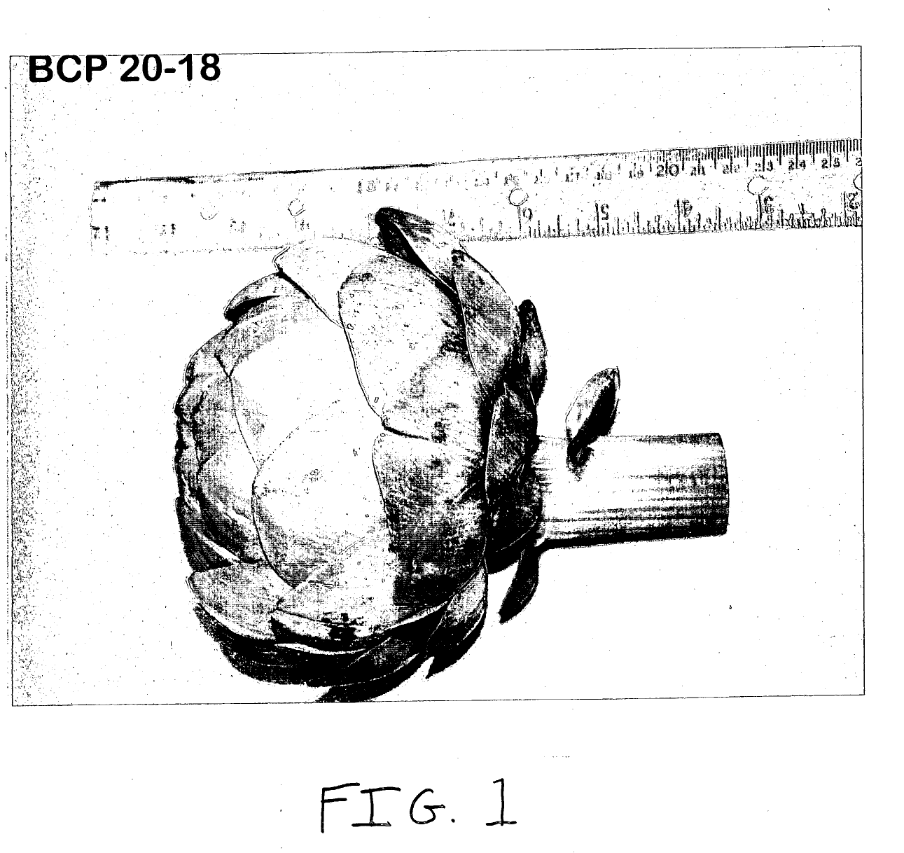

FIG. 2 shows a side elevational view of ‘BCP 20-18’s foliage in full color.

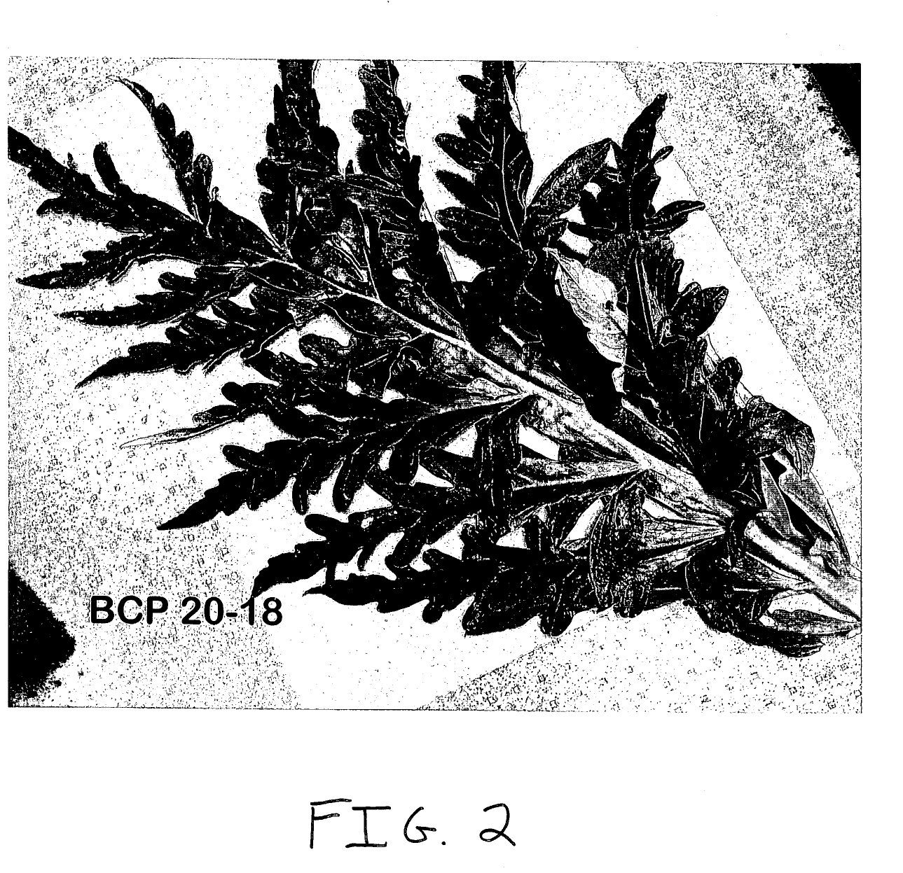

FIG. 3 shows a close-up view of the vertical cross-section of ‘BCP 20-18’s fruit in full color.

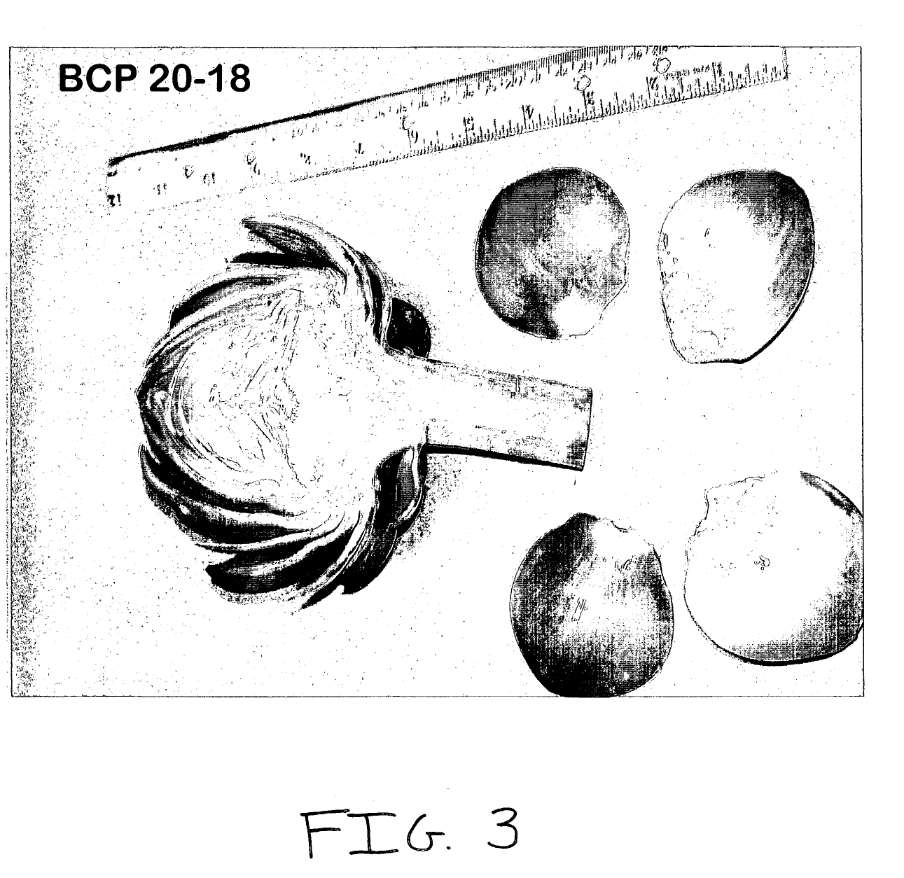

### DETAILED DESCRIPTION OF THE INVENTION

The following observations, measurements, and values describe the new artichoke plant is based upon observations of plants grown in Lompoc, Calif. The data was collected from tissue culture plants grown in Perpignan and acclimatized in California. Selections were made in outdoor field settings. The plants were grown in the soil in rows where row spacing (bed centers) was at 80 inches, and individual plant spacing at 24 inches. The plants were then planted in July and the observations described herein taken in December of the next year. Color references are measured against The Royal Horticultural Society Colour.

General:

Parentage.—Hybrid cross of BCL 9-11 and BH 35-28.

Classification.—Cynara Scolymus L. cv BCP 20-18.

Propagation.—Asexual production by division.

Plant:

Height.—106.7-121.9 cm; average 114.3 cm.

Width.—205.7-238.8 cm; average 226.1 cm.

Form.—Full.

Growth habit.—Upright.

Vigor.—Vigorous.

Side shoots:

Number per plant.—6.8.

Development.—Vigorous.

Color.—Green Group 139 c-d, highly pubescent.

Main stem diameter.—6.74-8.7 cm; average 7.8 cm.

Capitulum:

Primary size.—Approximately 13.3 cm.

Shape.—Compact, round, with pyramidal top.

Texture.—Hard and smooth.

Fragrance.—Mild.

Bract:

Length.—Approximately 7 cm.

Width.—Approximately 5.5 cm.

Shape.—Predominantly oval-shaped.

Texture.—Hard and smooth.

Number.—186 per head.

Color (inner).—Green group 141 d.

Color (outer).—Green group 141 d.

Firmness.—Firm and fleshy with thick basal thickness.

Spinosity.—None, or minimal — less than 1 mm.

Basal thickness.—6-17 mm.

Miscellaneous:

Heart description.—Concave full, flatter than Green Globe, well developed with thick bract connection.

Receptacle thickness.—1.6 cm.

Heart color.—Yellow green group 145b-c.

Florets.—Sterile.

Pappus length.—1.4 cm.

Pappus color.—Yellow green group 145d.

Head firmness.—Firm, heads are dense and solid.

Gloss.—Less glossy than Green Globe.

Cold storage (hold quality).—Good, heads remain firm and free of decay; slight leaf discoloration with cut stem turning brown. Stem was semi-soft after 7 days.

Head response (weather).—No adverse responses to weather conditions were observed on heads or other plant tissues more sensitive to freezes than red varieties.

Foliage:

Shape.—Long strap-like leaves with irregular dentate and very lobed.

Foliage density.—Dense; increased density observed with increased side shoot development.

Length.—Approximately 112.4-149.2 cm.

Width.—Approximately 71.1-94.0 cm.

Distance between main lobes.—Approximately 9.3-10.5 cm.

Leaf ratio.—Approximately 1.5-1.7 cm.

Leaf area.—Approximately 3,147-5,521 cm sq.

Upper leaf surface color.—Green group 137 a-b.

Lower leaf surface color.—Green group 136c.

Texture.—lightly but uniformly textured.

Pubescence.—Sparse, some visible pubescence on lower leaf surface.

Lobe width.—Approximately 13.3-24.8 cm.

Lobe indentation.—Approximately 9.5-19.7 cm.

Petiole length.—Approximately 33.0-38.1 cm.

Petiole width.—Approximately 3.2-7.4 cm.

Petiole thickness.—Approximately 2.2-3.2 cm.

Petiole color.—Green group 142 b-c.

Seeds:

Shape.—Ovoid asymmetrical.

Number.—Very irregular.

Length.—8-10 mm.

Width.—Not measured.

Color.—White yellow.

What is claimed is:

1. The new distinct variety of artichoke plant substantially as shown and described, characterized particularly as to novelty by the characteristics listed above.
